(19) United States
(12) Patent Application Publication
(10) Pub. No.: US 2023/0166052 A1
(43) Pub. Date: Jun. 1, 2023

(54) ENDOTHERMIC VAPOR AND ANTIMICROBIAL SKIN ANESTHETIC AND APPARATUS FOR APPLICATION

(71) Applicant: Vapocoolshot, Inc., Boca Raton, FL (US)

(72) Inventor: Jacob Leibovici, Wellington, FL (US)

(21) Appl. No.: 18/059,652

(22) Filed: Nov. 29, 2022

Related U.S. Application Data
(63) Continuation-in-part of application No. 17/537,424, filed on Nov. 29, 2021.

(51) Int. Cl. A61M 5/42; A61M 5/32; A61K 31/155

(52) U.S. Cl. CPC A61M 5/422; A61M 5/3287; A61K 31/155; A61M 2205/3606

(57) ABSTRACT

The present device provides a method and an apparatus for applying a vaporized endothermic gas to a skin surface to provide numbing of the application site for injection with a syringe. The device includes a container or canister containing an endothermic gas that rapidly absorbs heat when released to the atmosphere. A depressible actuating member or trigger propels the gas or vapor through an outlet nozzle that is constructed to break the liquid into a vapor that can be projected as a stream of vapor along a delivery axis that intersects a delivery axis of the syringe needle; therefore, the gas or vapor can be successively delivered to an injection site with minimal repositioning of the device.

### PRIORITY CLAIM

In accordance with 37 C.F.R. 1.76, a claim of priority is included in an Application Data Sheet filed concurrently herewith. Accordingly, the present invention claims priority as a Continuation-In-Part of U.S. patent application Ser. No. 17/537,424, entitled “APPARATUS FOR APPLYING AN ENDOTHERMIC VAPOR TO SKIN AS AN ANESTHETIC”, filed Nov. 29, 2021; and is related to U.S. patent application Ser. No. 16/017,379, entitled “METHOD AND APPARATUS FOR APPLYING AN ANESTHETIC AND BACTERICIDE”, filed Jun. 25, 2018, now U.S. Pat. No. 10,675,417, issued Jun. 9, 2020, which claims priority as a continuation-in-part of U.S. patent application Ser. No. 15/094,754, entitled “METHOD AND APPARATUS FOR APPLYING AN ANESTHETIC AND BACTERICIDE”, filed Apr. 8, 2016, now U.S. Pat. No. 10,004,855, issued Jun. 26, 2018, which claims priority to U.S. Provisional Patent Application No. 62/145,322, entitled “METHOD AND APPARATUS FOR APPLYING AN ANESTHETIC AND BACTERICIDE”, filed Apr. 9, 2015, which also claims priority as a continuation-in-part of U.S. patent application Ser. No. 14/453,475, entitled “METHOD AND APPARATUS FOR APPLYING AN ANESTHETIC AND BACTERICIDE”, filed Aug. 6, 2014, now U.S. Pat. No. 9,656,028, issued May 23, 2017, which is a continuation-in-part of U.S. Pat. No. 13,927,454, entitled “METHOD AND APPARATUS FOR APPLYING AN ANESTHETIC AND BACTERICIDE”, filed Jun. 26, 2013, now U.S. Pat. No. 9,561,334, issued Feb. 7, 2017, which is a continuation-in-part of U.S. patent application Ser. No. 12/557,753, entitled “METHOD AND APPARATUS FOR APPLYING AN ANESTHETIC”, filed Sep. 11, 2009, now U.S. Pat. No. 8,500,678, issued Aug. 6, 2013, which is a continuation-in-part of U.S. application Ser. No. 11/636,859, entitled “DENTAL SYRINGE”, filed on Dec. 11, 2006, now abandoned, which claims the priority to U.S. Provisional Patent Application No. 60/733,757, entitled “CRYO-SYRINGE”, filed on Mar. 7, 2006. The contents of which the above referenced applications are incorporated herein by reference.

### FIELD OF THE INVENTION

Generally, the invention relates to an apparatus for applying an endothermic gas to tissue to provide an anesthetic to an injection site prior to an injection to minimize the pain associated with conventional injection techniques. In particular, the apparatus comprises a receptacle for a liquefied endothermic gas and system for converting the liquefied endothermic gas to a vapor for application to the skin.

### BACKGROUND

Syringes are employed millions of times daily all over the world to inject medicines into people, as well as animals. Many times, injections are made in areas of the body that are somewhat less sensitive to pain. Other locations of the body where injections are contemplated are significantly more sensitive to pain, and the patient feels a pinching sensation that may be quite painful as the syringe needle is inserted beneath the skin. Such areas include, for example, gums and areas of the face, such as the forehead, as well as the lips. To minimize the pain that results when the injection needle penetrates, for example, a patient's gums, the dental practitioner will often apply a topical agent to the injection site using a cotton swab. Because the deadening agent is only applied topically, it is not effective, as it does not cross the skin/mucosal membranes and misleads the patient into a false expectation of a painless injection. As a result, injecting an anesthetic often causes significant pain at the injection site. In other cases, such as diabetics, patients may be required to self-medicate on a daily basis. The repeated injections often create sensitive areas where injections are painful to the patient. This pain may cause patients to delay or omit medication to avoid the pain associated therewith. Current devices, even those to the present inventor, which utilize cold sprays to numb the injection site suffer from applying the numbing agent as a liquid pressurized spray. The liquid spray fails to optimally atomize for proper cooling of the skin. The vaporization of the liquid is slowed, and thus the cooling effect is reduced for a given amount of spray applied.

Yet another issue associated with injection is infection. Diabetics are required to inject insulin at various intervals. This often requires the injection to be made at a less than optimal location which may not be completely sanitary. Insertion of the needle or the blade used to release blood for testing may cause microbes to be pushed under the dermis of the individual causing infection or worse. Thus, there is a need in the art for a numbing spray that also includes an antimicrobial to reduce the incidence of infection.

### SUMMARY

There is currently a need for a means of minimizing the pain associated with an injection. The present invention addresses this need by providing a syringe having a liquid compressed gas canister securely attached. The device includes an endothermic gas compressed to a liquid form. When the liquid endothermic material is released, it is converted to a vapor that can be directionally guided to the injection point to rapidly absorb heat when released to the atmosphere. The endothermic vapor is applied to the injection site prior to an injection to minimize the pain associated with conventional injection techniques. Furthermore, the vapor also blanches the mucosa almost instantly, allowing a practitioner to readily identify the pretreated injection site so that the needle is not inserted into an unanesthetized area.

Embodiments of the invention are also directed to an apparatus comprising a syringe which is removably attached to a cold spray module for converting the liquefied gas to a vapor by impingement. The cold spray module is secured to a compressed gas assembly which holds the liquefied endothermic gas. The apparatus comprises an actuating member which acts to dispense the contents of a container or canister containing the anesthetic composition. The cold spray module includes an actuating member for the controlled release of the liquefied endothermic gas for conversion to a vapor.

Embodiments of the invention are further directed to a liquefied endothermic gas canister assembly for attachment to the present device. The canister assembly includes a dispensing valve that is secured to a locking collar. The combination of the dispensing valve and the locking collar prevent other types of pressurized canisters from being secured to the dispensing valve for the present device, thereby reducing or preventing the present device from being used with unknown gaseous materials. The combination also prevents the gas cylinders from being refilled with the wrong compressed gas.

Other aspects are described infra.

### BRIEF DESCRIPTION OF THE DRAWINGS

The patent or application contains at least one drawing executed in color. Copies of this patent or patent application publication with color drawings will be provided by the Office upon request and payment of the necessary fee.

FIG. 1 is a perspective view of the cold spray assembly secured to the gas cylinder assembly ready for use;

FIG. 2 is a perspective view of the syringe module with the gas cylinder assembly detached;

FIG. 3 is a perspective view illustrating the gas cylinder assembly with the syringe module connected;

FIG. 4 is a partial exploded perspective view of the actuator portion of the gas cylinder assembly;

FIG. 5 is a section view of the actuator taken along lines 5-5 of FIG. 4;

FIG. 6A is a perspective view illustrating a core tube of the cold spray assembly securable to an outer surface of the gas cylinder outlet tube;

FIG. 6B is a perspective view illustrating a core tube of the cold spray assembly securable to an inner surface of the gas cylinder outlet tube;

FIG. 7A is a front view of the gas cylinder assembly;

FIG. 7B is a side view of the gas cylinder assembly of FIG. 7A;

FIG. 7C is a section view taken along lines 7C-7C of FIG. 7B;

FIG. 7D is an enlarged partial section view taken along lines 7D-7D of FIG. 7C illustrating the locking ring for providing a tamper resistant gas cylinder assembly;

FIG. 7E is a top view of the gas cylinder assembly of FIG. 7A;

FIG. 7F is a partially exploded view illustrating the locking ring of the gas cylinder assembly;

FIG. 7G is a partial perspective view of the gas cylinder assembly of FIG. 7A;

FIG. 8 is an exploded view of the gas cylinder assembly illustrated in FIGS. 7A-7G;

FIG. 9 is an alternative embodiment of the gas cylinder assembly constructed without the locking ring;

FIG. 10 is a section view of one embodiment of the cold spray assembly secured to a gas cylinder assembly;

FIG. 11 is an exploded view of the cold spray assembly of FIG. 10;

FIG. 12A is a perspective view of one embodiment of the core tube of the cold spray module;

FIG. 12B is a side view of the core tube of FIG. 12A;

FIG. 12C is a front view of the core tube of FIG. 12A;

FIG. 12D is a section view taken along lines 12D-12D of FIG. 12C;

FIG. 13A is a perspective view of the nozzle body for the cold spray module;

FIG. 13B is a side view of the nozzle body of FIG. 13A;

FIG. 13C is a front view of the nozzle body of FIG. 13A;

FIG. 13D is a section view taken along lines 13D-13D of FIG. 13C;

FIG. 14A is a front perspective view of a breakup nozzle for the cold spray module;

FIG. 14B is a rear perspective view of the breakup nozzle of FIG. 14A;

FIG. 14C is a side view of the breakup nozzle of FIG. 14A;

FIG. 14D is a side view of the breakup nozzle of FIG. 14A;

FIG. 14E is a front view of the breakup nozzle of FIG. 14A;

FIG. 14F is a rear view of the breakup nozzle of FIG. 14A;

FIG. 14G is a section view of the breakup nozzle of FIG. 14A taken along lines 14G-14G of FIG. 14C; and

FIG. 15 illustrates a spray sequence for the 80/20 SOLSTICE® Propellant: HFO-1234ze(E) and SOLSTICE® Enhance: HFO-1233zd(E) mixed with 10% or less of PERIDEX™ chlorhexidine gluconate 0.12%.

### DETAILED DESCRIPTION

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

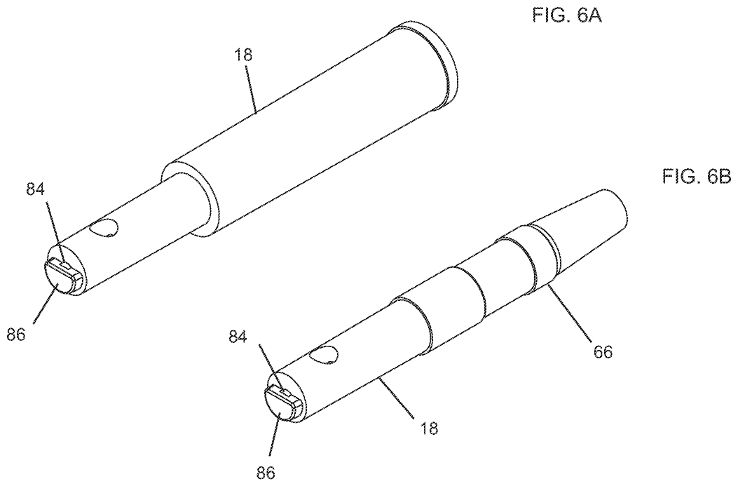

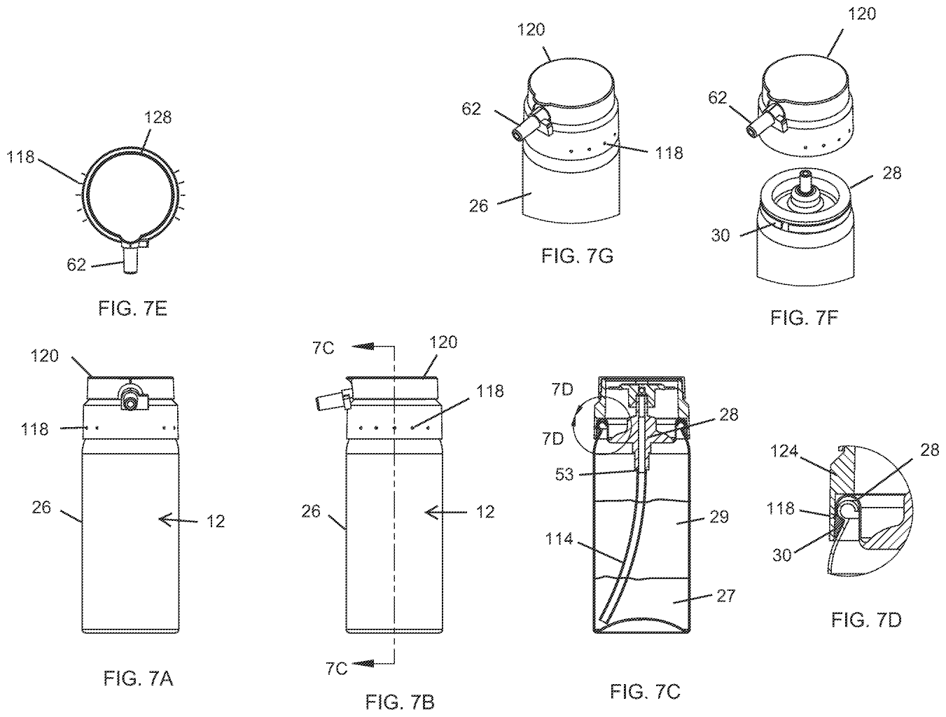

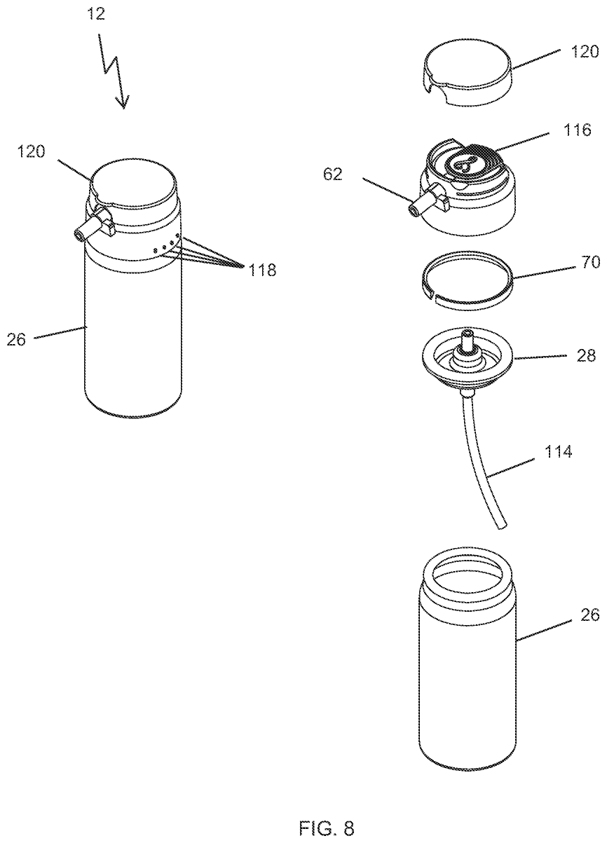

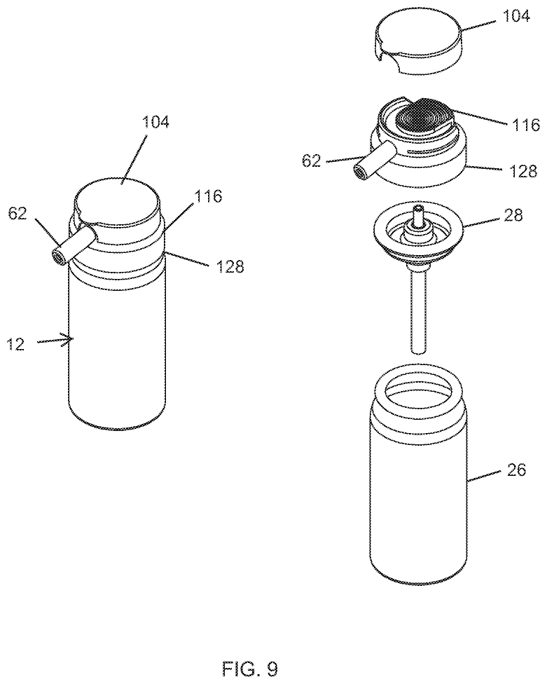

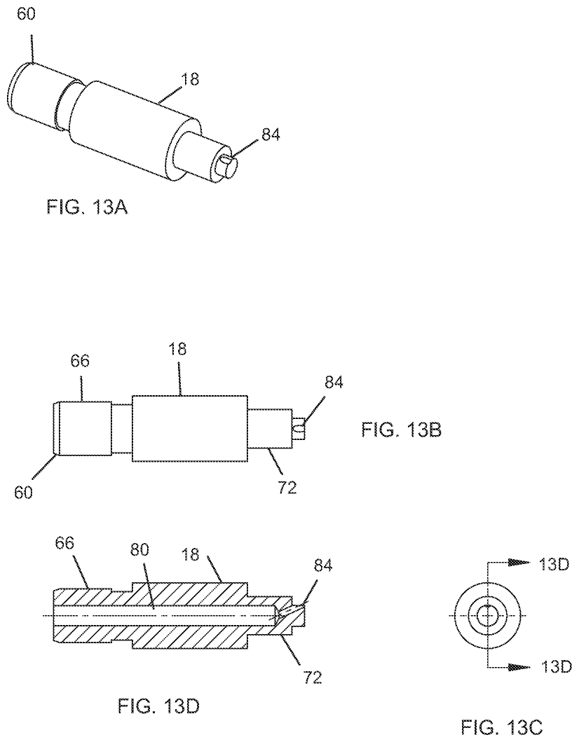

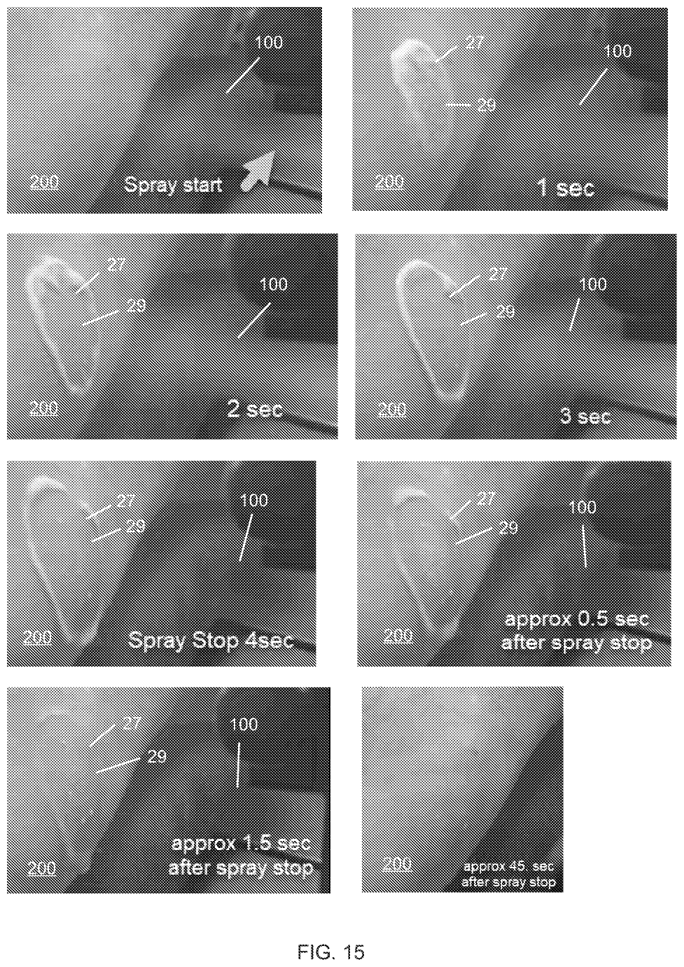

Referring to FIGS. 1-15, embodiments of the present invention relate to a method, anesthetic and apparatus of applying an anesthetic which may include an antimicrobial in the form of a vaporized endothermic gas 29 (FIG. 7C). The endothermic vapor applicator 100 in some embodiments is attachable to a syringe to provide the endothermic vapor along a trajectory that aligns with the puncture point of the syringe needle. This construction allows the user to align the device once for the skin 200 or epidermis, dermis numbing provided by the endothermic gas vapor as it cools the skin to be followed by the injection without the need for repositioning the device. The endothermic vapor applicator 100 includes a cold spray module 10, and a canister assembly 12. The cold spray module 10 includes a cold spray module housing 16, a core tube 18, and a breakup nozzle 20 which may be incorporated into the outlet. The canister assembly 12 includes a canister 26, a valve assembly 28, and a locking ring 30. When used, the syringe (not shown) includes a barrel, a plunger, a barrel tip and a needle.

Referring generally to the Figures, and more specifically to FIGS. 1-5, the endothermic vapor applicator 100 is illustrated as an assembly. In general, the cold spray module 10 is constructed and arranged for removable attachment to the canister assembly 12, as well as removably attached to the syringe to connect the assemblies together in a manner that allows the cold vapor to be controllably sprayed and the syringe to be positioned for operation of the cold spray module 10 and the syringe with one hand. In this manner, the endothermic vapor applicator 100 can be aimed at a desired injection site and the endothermic gas (29) released, for example, with the thumb or index finger. The thumb can then be repositioned to the plunger of the syringe for an injection. The endothermic gas vapor creates a blanched area on the skin 200, which is visible to insert a needle or finger stick into the numbed area. The ergonomic construction of the endothermic vapor applicator 100 allows both operations to occur without repositioning of the hand with respect to the cold spray module 10 and canister assembly 12 or visa-versa. Thus, the breakup nozzle 20 is constructed and arranged to cause the endothermic vapor to be directed as a stream to intersect with a delivery axis of the needle connected to the syringe when the syringe is attached to the cold spray module 10.

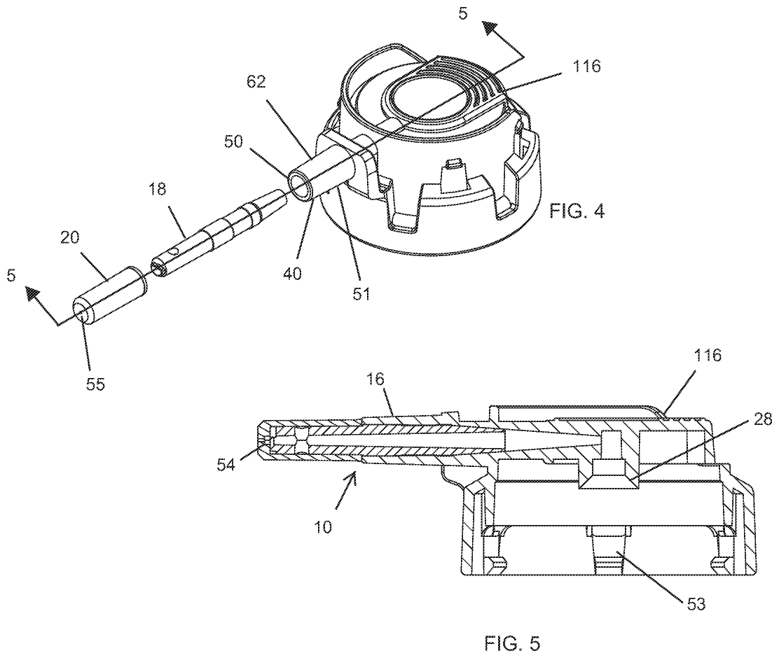

Referring to FIGS. 1-15, the cold spray module 10 is illustrated. The cold spray module 10 generally connects either permanently or removably to the canister assembly 12 and maintains the positioning of each assembly with respect to the other. In addition, the cold spray module 10 routes the liquefied endothermic gas from the canister assembly 12 to the desired area of the skin 200, breaking the liquid into a stream of vapor as it passes through the cold spray module 10. The cold spray module 10 includes a cold spray module housing 16 formed by a portion of the discharge tube 62, a core tube 18, and a breakup nozzle 20. The housing 16 is constructed generally as a tubular portion 40. The internal bore 50 of the tubular portion 40 is generally a smooth, consistent or slightly tapered bore and may contain one or more internal flanges 52 for maintaining the position of the core tube 18 and breakup nozzle 20 within the bore. Thus, the core tube 18 seen in FIGS. 4-5 and 6A-6B is inserted into the internal bore 50 or the outer diameter 51 of the tubular portion 40. The core tube 18 receives the liquefied endothermic gas from the canister assembly 12 and provides a pathway for the liquid to travel to a position where the liquid endothermic gas (29) is directed to impinge at an angle upon a breakup surface 54 of the breakup nozzle 20 to convert the liquid to a vapor. The vapor is thus expanded to cause it to escape through the orifice under pressure to create a stream of endothermic vapor the user can direct at a skin surface 200. In at least some embodiments, a siphon valve 53 is opened when the plunger 34 is depressed allowing either the liquid endothermic gas or a combination of the endothermic gas and the antimicrobial solution to be dispensed simultaneously. While in some embodiments, the antimicrobial (27) (FIG. 7C) has a higher density than the endothermic gas 29, and therefore rests in the bottom portion of the canister 26. Opening the siphon valve 53 allows the endothermic gas to escape through the valve, creating a low pressure area within the dip tube, drawing the antimicrobial liquid into the stream of endothermic gas, causing them both to be expelled at the same time, thereby numbing and sanitizing the numbed area of the skin 200.

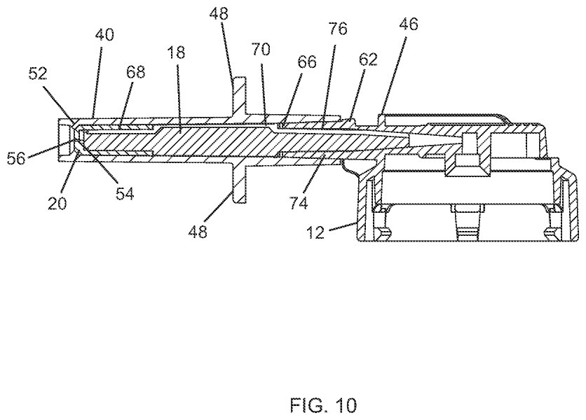

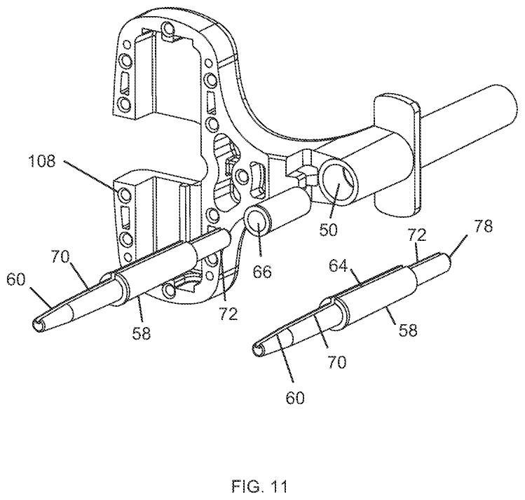

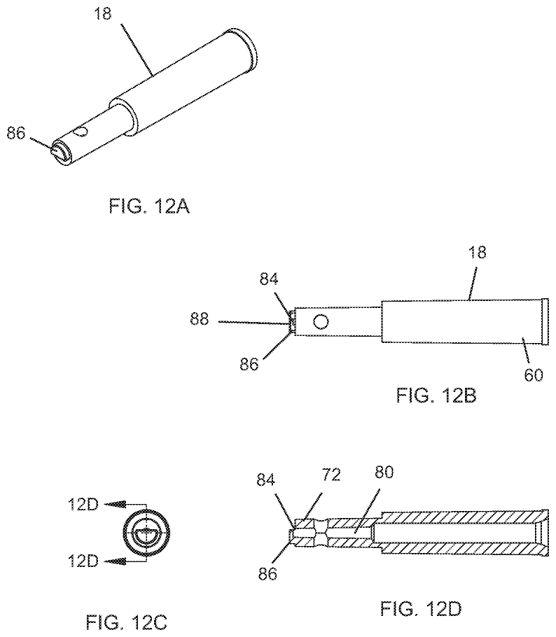

Referring generally to the Figures, and more specifically to FIGS. 10-14G, various embodiments of the core tube 18 are illustrated. In general, the core tube 18 is constructed and arranged to be in fluid communication with the canister assembly 12 to transfer the compressed liquid endothermic gas from the canister 26 and direct the liquid into the breakup surface 54 of the breakup nozzle 20. Thus, the core tube 18 may be provided in various forms to provide the same or similar functions. FIGS. 10-11 and 13 illustrate an embodiment of the core tube 18 that includes an elongated body 58 sized to fit snugly into the tubular portion 40 of the cold spray module 10. A first end 60 of the core tube 18 is constructed to fit snugly into a discharge tube 62 of the canister valve 28. A second end 72 is constructed to fit snugly into an inside bore 68 of the breakup nozzle 20. The snug fitment between the components allows the side surface 64 of the core tube 18 to include a U-shaped channel 70, whereby an internal surface 66 of the discharge tube 62, the internal bore 50 of the tubular portion 40 of the housing 16, and the inside bore 68 of the breakup nozzle 20 all form a closing surface for the U-shaped channel 70 to form a passage for the liquefied endothermic gas 29. It should be noted that the U-shaped channel 70 is positioned off center of the core tube 18, terminating in an annular groove 78 which directs the liquefied gas at a breakup surface 54 of the breakup nozzle 20. In this manner, the liquefied gas is required to impact the breakup surface 54 and travel across it at an angle to reach the orifice 56 of the nozzle 20. FIGS. 12A-12D illustrate an alternative embodiment of the core tube 18. In this embodiment, the first end 60 of the tube is constructed and arranged to cooperate with an outer surface 51 of the discharge tube 62. In at least one embodiment, the discharge port 84 is constructed by positioning a pair of offset flat panels 86 with a U-shaped notch 88 positioned in a central portion of the forward most flat panel 86. The offset positioning of the two panels 86 causes the liquid to exit the central conduit 80 at an angle with respect to the longitudinal centerline of the core tube 18. In some embodiments, see FIG. 10, a tapered sleeve 76 may be overmolded or press-fit onto an outer surface of the tubing 74 to provide a snug fit to the inner surface 66 of the discharge tube 62 of the canister assembly 12. It should be noted that the term “snug” does not require a liquid or air tight seal, so long as the preponderance of the liquid is transferred through the core tube 18 to the breakup nozzle 20.

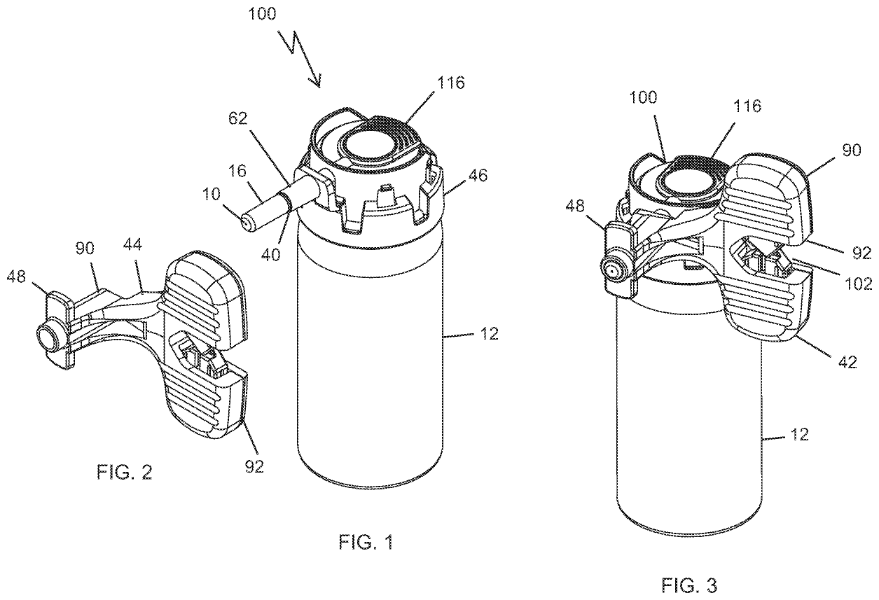

Referring generally to the Figures, and more specifically to FIGS. 2 and 3, wherein the cold spray module 10 includes a syringe barrel securing assembly 90; the syringe barrel securing assembly 90 including at least two opposing spring-loaded clamping members 92 configured for securing the syringe barrel to the cold spray module 10. Springs or the like may be placed into pockets 102 positioned in the ends of the clamping members 92 to bias the clamping members to a closed or clamping position. While coil springs are preferred, it should be noted that other biasing members, such as elastomeric, rubber or torsion members, may be utilized without departing from the scope of the invention. In a preferred embodiment, the springs are calibrated to retain a 1 to 3 cubic centimeter syringe while in other embodiments; the syringe may be as large as a 15 cubic centimeter syringe or larger.

Referring generally to the Figures, and more specifically to FIGS. 1, 3-5, 7A-9, embodiments of the canister assembly 12 are illustrated. The canister assembly 12 includes a metal canister 26 having a valve assembly 28 crimped and sealed to cover an open end of the canister 26 to create a sealed pressure canister. In general, the canister assembly 12 is constructed and arranged to contain the liquefied endothermic gas 29 sufficiently pressurized to maintain the liquid state of the gas until released from the canister 26. The valve assembly 28 includes a dispenser tube 114 extending outwardly therefrom for connection to the cold spray module 10 through a discharge tube 62 secured to a discharge actuator 116. The discharge actuator 116 is constructed and arranged to operate by depressing the dispenser tube 114 in the valve assembly 28, which allows the liquefied endothermic gas to flow through the valve assembly 28 and through the discharge tube 62. In one embodiment, a siphon valve 53 is opened when the valve assembly 28 is operated. The function of the siphon valve 53 is to allow the endothermic gas to be expelled at the same or similar rates as an antimicrobial agent through the valve 53. In this manner, the endothermic gas 29 numbs the desired area while the antimicrobial disinfects the numbed area. Because the antimicrobial is heavier in density than the endothermic gas, it will rest on the bottom area of the canister 26. In order to discharge both materials, the siphon valve 53 opens to allow the endothermic gas to be expelled through the discharge tube 62, causing a siphon effect on the dispenser tube 114, thereby drawing the antimicrobial solution from the bottom portion of the canister 26 and simultaneously dispensing both materials from a single container. In at least one embodiment, a locking ring 30 is placed below the valve assembly crimp utilized to attach the valve assembly 28 to the canister 26. The locking ring 30 is laser or radio frequency welded 118 to the discharge actuator 116 to prevent removal of the discharge actuator 116 from the canister 26. This construction prevents unwanted refilling of the canister 26, which may allow contaminants or incorrect liquids into the system. Removal of the discharge actuator 116 requires destruction of the locking ring 30 as an obvious indicator of refilling or tampering of the canister 26. A cap 120 is constructed and arranged to cooperate with the discharge actuator 116 to prevent inadvertent operation thereof. Examples of endothermic liquefied gasses that can be used include, but should not be limited to, SOLSTICE® Propellant: HFO-1234ze(E) and/or SOLSTICE® Enhance: HFO-1233zd(E) mixed with 10% or less by volume of PERIDEX™ chlorhexidine gluconate 0.12% as an antibacterial. The chlorhexidine gluconate is formed from 1,1¹ hexamethylene bis [5-(p-chlorophenyl) biguanide] di-D-gluconate) in a base containing water, 11.6% alcohol, glycerin, PEG-40 sorbitan diisostearate, flavor, sodium saccharin, and FD&C Blue No. 1. Peridex is a near-neutral solution (pH range 5-7). Chlorhexidine gluconate is a salt of chlorhexidine and gluconic acid. It should be noted that other combinations of liquefied endothermic gasses and materials, such as antibacterials, may be combined with the endothermic gasses to reduce the possibility of frostbite, while still reducing the temperature of the skin 200 to the level necessary to reduce the pain of the injection, as well as providing antibacterial function. It should be noted that while the preferred mixture having 10% or less by volume of PERIDEX™ chlorhexidine gluconate 0.12% as an antibacterial is not limited to this specific combination and may be any effective dose as recognized for use by a physician and medical experts. It should also be noted that the chlorhexidine gluconate may be added to the refrigerant to raise the effective temperature of the refrigerant to control frostbite of the skin. In this manner, higher volumes of chlorhexidine gluconate can be utilized to regulate the refrigerant and how it reduces the temperature of the skin. Adding the chlorhexidine gluconate causes the refrigerant to act partially on the antibacterial in the warm atmosphere thereby reducing the refrigerants ability to cool the skin.

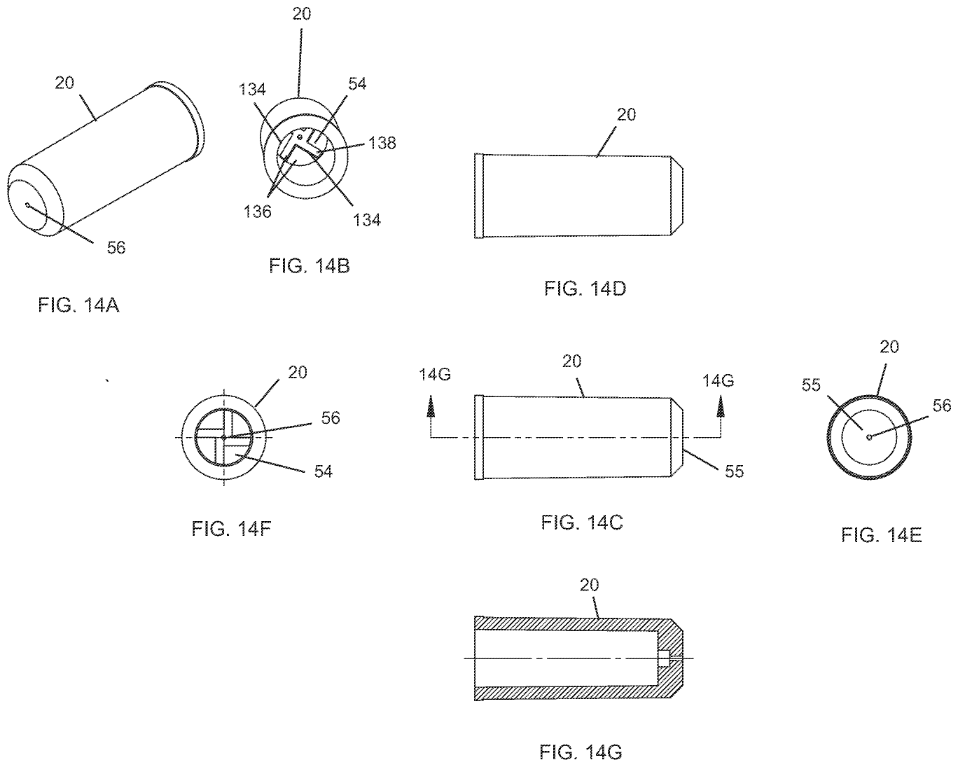

Referring generally to the Figures, and more specifically to FIGS. 14A-14G, an embodiment of the breakup nozzle 20 is illustrated. The breakup nozzle 20 includes a rear breakup surface 54, and a front surface 55 including the nozzle orifice 56. The rear breakup surface 54 includes at least one shearing corner 134, and more preferably a plurality of shearing corners 134. In general, the shearing corners 134 are sharp corners constructed from two adjoining planar surfaces 136, one of the planar surfaces 136 aligned parallel with respect to the longitudinal centerline of the core tube 18, and one of the planar surfaces 136 arranged perpendicular to the core tube 18. In this manner, the liquefied endothermic gas directed at the rear breakup surface 54 impinges the rear surface 54 adjacent the nozzle orifice 56, wherein the liquid breaks up and expands to exit the orifice 56 as a vapor with velocity. In a most preferred embodiment, the at least one shearing corner 134 is a sharp corner constructed from three adjoining planar surfaces 136, constructing a U-shaped breaker channel 138, two of the planar surfaces 136 aligned parallel with respect to the longitudinal centerline of the core tube 18, and one of the planar surfaces 136 arranged perpendicular to the longitudinal centerline of the core tube 18. In another embodiment, the rear surface 54 of the breakup nozzle 20 includes four of the U-shaped breaker channels 138. The breaker channels 138 may be arranged at right angles with respect to each other, breaking the rear surface 54 into four quadrants. The core tube 18 is constructed and arranged to direct the liquefied endothermic gas at the rear breakup surface 54 at an acute angle with respect to the longitudinal centerline of the core tube 18 to break the liquefied endothermic gas into a vapor which is directed at a skin surface to decrease the temperature of the skin suitably to cause numbness of the skin. Because of its physical properties, the heat-absorbing endothermic vapor constricts blood flow of the skin 200 at the injection site, and temporary numbing occurs. The endothermic vapor stops the propagation of the painful nerve stimuli, and the patient feels the tactile, or pressure, as opposed to the pain sensation. According to the Gate theory, the pressure nerve fibers supersede the painful nerve fibers so that the mechanical contraction of the muscles blocks the transmission of pain perception. The use of the endothermic vapor also temporarily distracts the patient by creating a noise that diverts the patient's attention away from any potential or anticipated pain. Finally, because the endothermic vapor blanches the mucosa, a readily-visible target is created for insertion of the needle to assure that the deadened area is not bypassed. When the practitioner observes that the injection site mucosa has been blanched, the site is effectively deadened and a painless, concomitant injection is possible. The practitioner can then quickly inject the injection site by inserting the injection needle and depressing the discharge actuator 116. Because of the positioning of the breakup nozzle 20 with respect to the needle, the dispersal of the vapor and subsequent injection of anesthetic can be accomplished almost concurrently, and with no pain to the patient.

Referring generally to the Figures, and more specifically to FIGS. 2-3, an embodiment of the present device that includes a syringe barrel securing assembly 90 is illustrated. The syringe barrel securing assembly 90 includes a pair of finger grips 42. In some embodiments, a positioning anchor 44 extends from the area between the tubular portion 40 and the syringe barrel securing assembly 90 to snap onto the top rim 46 of the valve assembly 28 to prevent rotation and linear translation between the canister assembly 12 and the cold spray module 10. Secondary finger grips 48 may be integrally formed to the tubular portion 40 to provide versatility to gripping the endothermic vapor applicator 100.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An endothermic vapor applicator (100) comprising:
a canister assembly (12) containing a liquefied endothermic gas (29), the canister assembly (12) fluidly connected to the cold spray module (10) so that the liquefied endothermic gas is broken up into droplets prior to discharge from the cold spray module (10), the canister assembly (12) including an antimicrobial liquid mixed with the endothermic gas within the canister (26), wherein the liquefied endothermic gas (29) causes the antimicrobial liquid (27) to be entrained within the liquefied endothermic gas (29) as it is expelled from the canister (26), the liquefied endothermic gas (29) being constructed and arranged to have a suitable temperature to cause numbness when directed at human skin (200) without causing frostbite, the antibacterial liquid (27) disinfecting the numbed area.

2. The endothermic vapor applicator (100) of claim 1 wherein the liquefied endothermic gas (29) is HFO-1234ze(E).

3. The endothermic vapor applicator (100) of claim 2 wherein the antibacterial liquid (27) is chlorhexidine gluconate.

4. The endothermic vapor applicator (100) of claim 2 wherein the antibacterial liquid (27) is chlorhexidine gluconate 0.12%.

5. The endothermic vapor applicator (100) of claim 1 wherein the endothermic gas (29) is HFO-1233zd(E).

6. The endothermic vapor applicator (100) of claim 5 wherein the antibacterial liquid (27) is chlorhexidine gluconate.

7. The endothermic vapor applicator (100) of claim 5 wherein the antibacterial liquid (27) is chlorhexidine gluconate 0.12%.

8. The endothermic vapor applicator (100) of claim 1 wherein the endothermic gas (29) is a combination of HFO-1234ze(E) and HFO-1233zd(E).

9. The endothermic vapor applicator (100) of claim 8 wherein the antibacterial liquid (27) is chlorhexidine gluconate.

10. The endothermic vapor applicator (100) of claim 8 wherein the antibacterial liquid (27) is chlorhexidine gluconate 0.12%.

11. The endothermic vapor applicator (100) of claim 1 wherein the canister assembly (12) includes a cold spray module (10), the cold spray module (10) including a core tube (18) for directing the liquefied endothermic liquid (29) to impinge against a breakup surface (54) for breaking the liquid stream into an endothermic vapor before the endothermic vapor is directed through a breakup nozzle (20), the breakup nozzle (20) is constructed and arranged to cause the endothermic vapor to be directed as a stream.

12. The endothermic vapor applicator (100) according to claim 1 wherein the canister (26) is a metal canister having a valve assembly (28) crimped and sealed within an open end of the canister (26) to create a sealed pressure canister, the valve assembly (28) including a discharge tube (62) extending outwardly therefrom for connection to the cold spray module (10).

13. The endothermic vapor applicator (100) according to claim 12 wherein the valve assembly (28) is finger operable to release the liquefied endothermic gas (29).

14. The endothermic vapor applicator (100) according to claim 1 wherein the breakup surface (54) is a rear surface of the breakup nozzle (20), the liquefied endothermic gas (29) directed at the breakup surface (54) to impinge the breakup surface (54) adjacent a nozzle orifice (56), wherein the liquefied endothermic gas (29) breaks up and expands to exit the nozzle orifice (56) with velocity.

15. The endothermic vapor applicator (100) according to claim 14 wherein the breakup surface (54) of the breakup nozzle (20) includes at least one shearing corner (134).

16. The endothermic vapor applicator (100) according to claim 14 wherein the breakup surface (54) of the breakup nozzle (20) includes a plurality of shearing corners (134).

17. The endothermic vapor applicator (100) according to claim 14 wherein the at least one shearing corner (134) is a sharp corner constructed from two adjoining planar surfaces (136), one of the planar surfaces (136) aligned parallel with respect to the longitudinal centerline of the core tube (18), and one of the planar surfaces (136) arranged perpendicular to the core tube (18).

18. The endothermic vapor applicator (100) according to claim 14 wherein the at least one shearing corner (134) is a pair of sharp corners constructed from three adjoining planar surfaces (136), constructing a U-shaped channel (70), two of the planar surfaces (136) aligned parallel with respect to the longitudinal centerline of the core tube (18), and one of the planar surfaces (136) arranged perpendicular to the core tube (18).

19. The endothermic vapor applicator (100) according to claim 14 wherein the core tube (18) is constructed and arranged to direct the liquefied endothermic gas (29) at the breakup surface (54) of the breakup nozzle (20) at an acute angle with respect to the longitudinal centerline of the core tube (18).

20. The endothermic vapor applicator (100) according to claim 14 wherein a discharge actuator (116) is constructed and arranged to operate by depressing a dispenser tube (114) in the valve assembly (28), which allows the liquefied endothermic gas to flow through the valve assembly (28) and through the discharge tube (62).

21. The endothermic vapor applicator (100) according to claim 20 wherein a siphon valve (53) is opened when the valve assembly (28) is operated, the siphon valve (53) causes the endothermic gas to be expelled at the same or similar rates as the antimicrobial agent through the valve (53), the antimicrobial having a higher density than the endothermic gas, causing it to be drawn through the dispenser tube (114) while the preponderance of the endothermic gas is passed through the siphon valve (53), causing both materials to be expelled through the discharge tube (62).
